SEQUENCE LISTING (18 sequences)

SEQ ID NO: 1 - protein, 112 aa, Homo sapiens
ELVMTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKR

SEQ ID NO: 2 - protein, 135 aa, Homo sapiens
LEQSGAELVKPGASVKISCKASGYSFTDYNMNWVKQSNGKSLEWIGVINPNSGTTGYNQKFKGKATLTVDQSSSTAYMQLNSLTSEDSAVYYCAEYYYGEDPFAYWGQGTLVTVSTAKTTPPSVYPLAPGSAAQT

SEQ ID NO: 3 - protein, 18 aa, Homo sapiens
GSTSGSGKPGSGEGSTKG

SEQ ID NO: 4 - protein, 247 aa, Homo sapiens
LEQSGAELVKPGASVKISCKASGYSFTDYNMNWVKQSNGKSLEWIGVINPNSGTTGYNQKFKGKATLTVDQSSSTAYMQLNSLTSEDSAVYYCAEYYYGEDPFAYWGQGTLVTVSTAGSTSGSGKPGSGEGSTKGELVMTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKR

SEQ ID NO: 5 - protein, 247 aa, Homo sapiens
ELVMTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKRGSTSGSGKPGSGEGSTKGLEQSGAELVKPGASVKISCKASGYSFTDYNMNWVKQSNGKSLEWIGVINPNSGTTGYNQKFKGKATLTVDQSSSTAYMQLNSLTSEDSAVYYCAEYYYGEDPFAYWGQGTLVTVSTA

SEQ ID NO: 6 - protein, 15 aa, Homo sapiens
EPKSPKSADKTHTAP

SEQ ID NO: 7 - protein, 101 aa, Homo sapiens
PAAPQVYTIPPPLEQMAKDLVSLTCMITDFFPEDITVEWQWNGQPAENYKNTQPIMDTDGSYFVYSKLNVQKSNWEAGNTFTCSVLHEGLHNHHTEKSLSH

SEQ ID NO: 8 - protein, 116 aa, Homo sapiens
EPKSPKSADKTHTAPPAAPQVYTIPPPLEQMAKDLVSLTCMITDFFPEDITVEWQWNGQPAENYKNTQPIMDTDGSYFVYSKLNVQKSNWEAGNTFTCSVLHEGLHNHHTEKSLSH

SEQ ID NO: 9 - DNA, 1089 bp, Homo sapiens
  source 1..1089: mol_type = genomic DNA
ctcgagcagtctggagctgagctggtgaagcctggcgcttcagtgaagatatcctgcaaggcttctggttactcattcactgactacaacatgaactgggtgaagcagagcaatggaaagagccttgagtggattggagtaattaatcctaactctggtactactggctacaatcagaagttcaagggcaaggccacattgactgtagaccaatcttccagcacagcctacatgcagctcaacagcctgacatctgaggactctgcagtctattactgtgcagaatattactacggcgaggatccttttgcttactggggccaagggactctggtcactgtctctacagccggcagcaccagcggcagcggcaaaccgggcagcggcgaaggcagcaccaaaggcgaactcgtgatgacacagtctcctgcttccttagctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaacgggaaccgaaaagcccgaaaagcgcggataaaacccataccgcgccgccggcggctccacaggtgtacaccattccacctcccctggagcagatggccaaggatctagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccac

SEQ ID NO: 10 - protein, 363 aa, Homo sapiens
LEQSGAELVKPGASVKISCKASGYSFTDYNMNWVKQSNGKSLEWIGVINPNSGTTGYNQKFKGKATLTVDQSSSTAYMQLNSLTSEDSAVYYCAEYYYGEDPFAYWGQGTLVTVSTAGSTSGSGKPGSGEGSTKGELVMTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKREPKSPKSADKTHTAPPAAPQVYTIPPPLEQMAKDLVSLTCMITDFFPEDITVEWQWNGQPAENYKNTQPIMDTDGSYFVYSKLNVQKSNWEAGNTFTCSVLHEGLHNHHTEKSLSH

SEQ ID NO: 11 - DNA, 1089 bp, Homo sapiens
  source 1..1089: mol_type = genomic DNA
gagctcgtgatgacacagtctcctgcttccttagctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaacggggcagcaccagcggcagcggcaaaccgggcagcggcgaaggcagcaccaaaggcctcgagcagtctggagctgagctggtgaagcctggcgcttcagtgaagatatcctgcaaggcttctggttactcattcactgactacaacatgaactgggtgaagcagagcaatggaaagagccttgagtggattggagtaattaatcctaactctggtactactggctacaatcagaagttcaagggcaaggccacattgactgtagaccaatcttccagcacagcctacatgcagctcaacagcctgacatctgaggactctgcagtctattactgtgcagaatattactacggcgaggatccttttgcttactggggccaagggactctggtcactgtctctacagccgaaccgaaaagcccgaaaagcgcggataaaacccataccgcgccgccggcggctccacaggtgtacaccattccacctcccctggagcagatggccaaggatctagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccac

SEQ ID NO: 12 - protein, 362 aa, Homo sapiens
ELVMTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKRGSTSGSGKPGSGEGSTKGLEQSGAELVKPGASVKISCKASGYSFTDYNMNWVKQSNGKSLEWIGVINPNSGTTGYNQKFKGKATLTVDQSSSTAYMQLNSLTSEDSAVYYCAEYYYGEDPFAYWGQGTLVTVSTAEPKSPKSADKTHTAPPAAPQVYTIPPPLEQMAKDLVSLTCMITDFFPEDITVEWQWNGQPAENYKNTQPIMDTDGSYFVYSKLNVQKSNWEAGNTFTCSVLHEGLHNHHTEKSLS

SEQ ID NO: 13 - protein, 10 aa, Homo sapiens
QSVSTSSYSY

SEQ ID NO: 14 - skipped

SEQ ID NO: 15 - protein, 7 aa, Homo sapiens
HSWEIPY

SEQ ID NO: 16 - protein, 8 aa, Homo sapiens
GYSFTDYN

SEQ ID NO: 17 - protein, 8 aa, Homo sapiens
INPNSGTT

SEQ ID NO: 18 - protein, 12 aa, Homo sapiens
AEYYYGEDPFAY

(19) United States
(12) Patent Application Publication
(10) Pub. No.: US 2025/0026857 A1
(43) Pub. Date: Jan. 23, 2025

(54) MINI BODIES AS VACCINES FOR HIV

(71) Applicant: POMONA RICERCA S.R.L., TORINO (IT)

(72) Inventors: Massimo CLEMENTI, Milano (IT); Roberto BURIONI, Segrate MI (IT); Roberta Antonia DIOTTI, Verdellino BG (IT)

(73) Assignee: POMONA RICERCA S.R.L., TORINO (IT)

(21) Appl. No.: 18/714,884

(22) PCT Filed: Nov. 29, 2022
(86) PCT No.: PCT/IB2022/061516

§ 371 Date: May 30, 2024

(30) Foreign Application Priority Data
Dec. 1, 2021 (IT) 102021000030398

(51) Int. Cl. C07K 16/42; A61K 39/00; A61P 37/04

(52) U.S. Cl. CPC C07K 16/4241; A61P 37/04; A61K 2039/505; C07K 2317/20; C07K 2317/622

(57) ABSTRACT

The present invention relates to an anti-idiotype minibody capable of specifically binding the idiotype of a human anti-gp120 antibody and capable of evoking an anti-gp120 immune response. The present invention also relates to immunogenic compositions including such minibody and the use thereof in the therapeutic or prophylactic treatment of HIV infection or related diseases.

### TECHNICAL FIELD OF THE INVENTION

The present invention relates to an anti-idiotype minibody capable of specifically binding the idiotype of a human anti-gp120 antibody and capable of evoking an anti-gp120 immune response. The present invention also relates to immunogenic compositions comprising such minibody and the use thereof in the therapeutic or prophylactic treatment of HIV infection or related diseases.

### PRIOR STATE OF ART

Over time, the impact of HIV infection has reached enormous proportions. The impossibility of actuating infection control mechanisms, or the ineffectiveness thereof, already determined very serious consequences in different parts of the world. The antiretroviral therapy, if available, improved the life expectancy of the infected subjects. However, it is connected to a series of adverse factors which within few years could reduce the positive impact thereof. First of all, the currently available therapies do not succeed in removing completely the virus, but the patients remain infect and then always exposed to the risk of developing serious clinical forms of infection. Moreover, the available drugs frequently induce side effects. In addition, the high cost of therapy makes its use absolutely improbable on large scale in the depressed areas in the world which represent the real infection reservoir worldwide. As a whole, the above-described factors make it necessary to process alternative, or complementary, strategies. Moreover, for controlling HIV infection, the development of an effective vaccine approach will surely have a priority role. Unfortunately, the HIV infection still nowadays is an open challenge, difficult to be solved by the scientific community. To say the truth, the traditional approaches to vaccines, based upon the administration of viral particles which are not capable of infecting but which are capable of stimulating the immune system, demonstrated to be wholly ineffective against a virus which uses molecular polymorphism, that is the capability of changing to avoid the immune response, as winning weapon (McMichael J., (2006) Annu. Rev. Immunol. 24: 227-55). Other strategies for developing vaccines for controlling HIV infection resulted to be ineffective too. In the light of what said, the problem of the lack of a vaccine for HIV which is really effective is still of primary importance. In the patent EP2121763 (B1) the inventors, authors of the present invention, described an anti-immunoglobulin monoclonal antibody, capable of binding to the idiotype of anti-gp120 human antibodies. This monoclonal antibody, which can be defined as “anti-idiotype antibody, is capable of evoking a neutralizing anti-gp120 immune response in rabbits.

### SUMMARY OF THE INVENTION

In the light of an extensive experimentation performed by the inventors and described in details in the herein reported experimental section, it was surprisingly found that an antibody in minibody (mini-antibody) format directed against the idiotype of human anti-gp120 antibodies is capable of evoking an anti-gp120 immune response. The minibody format with respect to the complete immunoglobulin has several advantages, in particular in terms of pharmacokinetics.

A first object of the present invention is an anti-idiotype minibody comprising:
a single chain fragment variable (scFv) sequence capable of specifically binding the idiotype of a human anti-gp120 antibody, wherein said scFv comprises the variable domain of the heavy chain (VH) linked to the variable domain of the light chain (VL) with a linker sequence;
a hinge sequence;
a IgG CH3 domain.

An additional object of the present invention is the minibody or a sequence encoding said minibody or a vector comprising said sequence according to any one of the herein described embodiments for use in the therapeutic or prophylactic treatment of HIV infection or related diseases, in particular by inducing a neutralizing immune response against HIV.

An immunogenic composition comprising the minibody or a sequence encoding said minibody or a vector comprising said sequence according to any one of the herein described embodiments and a pharmaceutically acceptable carrier and/or diluent and/or adjuvant.

An additional object is a nucleotide sequence encoding the minibody according to any one of the herein described embodiments or an expression vector comprising said sequence, wherein said sequence or said vector are for use in the therapeutic or prophylactic treatment of HIV infection or related diseases.

The advantages, features and use modes of the present invention will result evident from the following detailed description of some embodiments, shown by way of example and not for limitative purposes.

### BRIEF DESCRIPTION OF FIGURES

FIG. 1. Affinity curve of b12 antibody, in Fab format, for P1 minibody with VH-VL orientation. Graph obtained from data obtained in ELISA.

FIG. 2. Affinity curve of b12 antibody, in Fab format, for P1 minibody with VL-VH orientation. Graph obtained from data obtained in ELISA.

FIG. 3. Graphic representation of the values obtained by gp120 ELISA assay of the not diluted serum of the different rabbits immunized with P1 minibody with VH-VL orientation.

FIG. 4. Graphic representation of the values obtained by gp120 ELISA assay of the not diluted serum of the different rabbits immunized with P1 minibody with VL-VH orientation.

FIG. 4B. Graphic representation of the values obtained by gp120 ELISA assay of the not diluted serum of the different rabbits immunized with negative minibody.

FIG. 5. Graphic representation of the values obtained by gp120 ELISA assay of the serum diluted 1:20 FIG. 5(A) and 1:200 FIG. 5(B) of the different rabbits immunized with P1 minibody with VH-VL orientation.

FIG. 6. Graphic representation of the values obtained by gp120 ELISA assay of serum diluted 1:20 FIGS. 6 (A) and 1:200 FIG. 6 (B) of the different rabbits immunized with P1 minibody with VL-VH orientation.

FIG. 7. Graphic representation of the values obtained by gp120 ELISA assay of serum diluted 1:20 FIGS. 7 (A) and 1:200 FIG. 7 (B) of the different rabbits immunized with negative minibody.

FIG. 8. Graphic representation of the values obtained on gp120/HIV by ELISA assay of the various dilutions of serum pre-and post-immunization of the cohort of rabbits immunized with P1 minibody with VH-VL orientation without having subtracted the signal obtained on BSA FIG. 8 (A) and after having subtracted it FIG. 8 (B). The graphs also show the p-values obtained by performing Wilcoxon test.

FIG. 9. Graphic representation of the values obtained on gp120/HIV by ELISA assay of the various dilutions of serum pre- and post-immunization of the cohort of rabbits immunized with P1 minibody with VL-VH orientation without having subtracted the signal obtained on BSA FIG. 9 (A) and after having subtracted it FIG. 9 (B). The graphs also show the p-values obtained by performing Wilcoxon test.

FIG. 10. Graphic representation of the values obtained on gp120/HIV by ELISA assay of the various dilutions of serum pre- and post-immunization of the cohort of rabbits immunized with negative minibody without having subtracted the signal obtained on BSA FIG. 10 (A) and after having subtracted it FIG. 10 (B). The graphs also show the p-values obtained by performing Wilcoxon test.

FIG. 11. Graphic representation of the values obtained on gp120/HIV, after having subtracted the signal on BSA, by ELISA assay of the various dilutions of serum post-immunization of the cohort of rabbits immunized with P1 minibody with VH-VL orientation, P1 minibody in VL-VH orientation and negative minibody. The graphs also show the p-values obtained by performing Mann-Withney test.

### DETAILED DESCRIPTION OF THE INVENTION

The present invention relates to an anti-idiotype minibody comprising:
a scFV sequence capable of specifically binding the idiotype of a human anti-gp120 antibody, wherein said scFv comprises a variable heavy chain (VH) linked to a variable light chain (VL) with a linker sequence;
a hinge sequence;
a IgG CH3 domain.

The term “idiotype” relates to all hypervariable regions of the variable domain of an immunoglobulin, that is those structures characterizing a homogeneous population of molecules of antibodies, such as for example the proteins of a myeloma or a monoclonal antibody, and then allowing to distinguish between a homogeneous population of antibody molecules and another homogeneous population (for example, between a monoclonal antibody and another one).

Under the expression “capable of specifically binding the idiotype of a human anti-gp120 antibody” it is meant that the minibody of the present invention is capable to react with the idiotype of a human antibody directed against the HIV gp120 protein (Envelope glycoprotein GP120). The minibody affinity for the idiotype could be measured in a direct affinity assay, or by measuring the capability of inhibiting the antibody bond with the gp120 protein by using assays known to the person skilled in the art.

The term scFv relates to an antibody format wherein the variable domains of the heavy chain (VH) and light chain (VL) of a traditional whole antibody were joined by a linker sequence, so as to form a single-chain variable fragment.

The minibody (mini-antibody) is an antibody format having a lower molecular weight (˜80 kDa) than the whole antibody (˜150 kDa), but it keeps the property of bivalent bond against a determined target (the minibody is described in Hu et al 1996 herein incorporated by reference). The “minibody” is a homodimer, wherein each monomer is a single-chain variable fragment (scFv) linked to a IgG1 CH3 domain by a hinge sequence.

The scFv can have a VH-VL or VL-VH orientation, wherein a VH-VL orientation means that the variable domain of the heavy chain (VH) of scFv is upstream of the variable domain of the light chain (VL) and a VL-VH orientation means that VL of scFv is upstream of VH. As used herein, “upstream” designates the direction of N-terminal of an amino acid sequence or the direction of the end 5′ of a nucleotide sequence. VH and VL are linked to each other by a linker sequence of amino acids, or of the nucleotide sequence encoding the minibody. As it results from the data reported in the experimental section the inventors surprisingly found that only the VH-VL configuration is capable of evoking an immune response directed against gp120/HIV statistically significative with respect to the one which develops by immunizing a control negative minibody. Therefore, the minibody preferred embodiments are those with VH-VL orientation.

The minibody according to the present invention results to be particular advantageous for its capability of stimulating a specific anti-gp120 immune response in animal models different from rats, for example in rabbits. The immunization experiments described in the experimental section of the patent application show that these minibodies, in particular those facing against the idiotype of the human b12 antibody, are capable of evoking a quick and strong specific anti-gp120 immune response. The obtained data then show that the herein described minibodies are particularly useful as vaccines, in particular for the prophylactic and therapeutic treatment of HIV infection or related diseases.

Therefore, the present invention relates to minibodies capable of reacting specifically with the idiotype of human anti-gp120 antibodies, in particular the present invention relates to minibodies capable of reacting specifically with the idiotype of human anti-gp120 antibodies directed against the gp-120 portion that binds the CD4 receptor, or the portions of the gp120 viral protein that bind the receptor of the target cell and the coreceptors at time of infection. For example, the present invention relates to minibodies capable of specifically binding the idiotype of the human b12 antibody described in Burton et al. ‘A large array of human monoclonal antibodies to type 1 human immunodeficiency virus from combinatorial libraries of asymptomatic seropositive individuals’ Proc. Natl. Acad. Sci. USA 88:10134-10137 and Burton et al. 1994. ‘Efficient neutralization of primary isolates of HIV-1 by a recombinant human monoclonal antibody’ Science 266:1024-1027 herein incorporated by reference.

According to an embodiment the minibody comprises at least a single-chain variable fragment (scFv), preferably both of them, wherein the domain of the heavy chain (VH) and the variable domain of the light chain (VL) respectively correspond to the sequences SEQ ID NO:2 and SEQ ID NO:1. According to an embodiment VH and VL correspond to those of P1 antibody described in the patent EP2121763 herein fully incorporated by reference. According to an embodiment VH and VL have a sequence identical at least by 95%, preferably at least by 98%, still more preferably by 99% with respect to the sequences SEQ ID NO:2 and SEQ ID NO:1 or to the VH and VL sequences of P1 antibody described in patent EP2121763.

According to an aspect of the invention, the variable domain of the heavy chain (VH) and the variable domain of the light chain (VL) to form single-chain variable fragment (scFv) will be joined by a linker with sequence SEQ ID NO:3. According to an embodiment the single-chain variable fragment (scFv) of the minibody is linked to the IgG1 CH3 domain with a hinge sequence having sequence SEQ ID NO:6.

According to an embodiment the IgG1 CH3 domain of the minibody is a murine CH3 domain, the CH3 domain of the minibody of the present invention for example could have the murine sequence SEQ ID NO:7.

According to an embodiment the minibody comprises at least one scFv, preferably both scFvs having SEQ ID NO:4 or SEQ ID NO:5. According to a preferred embodiment the minibody comprises two scFvs having SEQ ID NO:4 having VH-VL configuration. According to an embodiment at least one scFv, preferably both scFvs of the minibody have a sequence identical at least by 95%, preferably at least by 98%, still more preferably by 99% to the sequence SEQ ID NO:4.

According to an embodiment the minibody comprises at least one scFv, preferably both scFvs, wherein VH and VL have the following CDRs (Complementary Determining Region, based upon the IMGT database and/or IgBlast tool):
SEQ ID NO:13 Amino acid sequence CDR1 light chain (VL)
SEQ ID NO:14 Amino acid sequence CDR2 light chain (VL) (sequence YAS-Tyr-Ala-Ser)
SEQ ID NO:15 Amino acid sequence CDR3 light chain (VL)
SEQ ID NO:16 Amino acid sequence CDR1 heavy chain (VH)
SEQ ID NO:17 Amino acid sequence CDR2 heavy chain (VH)
SEQ ID NO:18 Amino acid sequence CDR3 heavy chain (VH)

According to the embodiments described in the present document, each monomer of the minibody can be encoded by a nucleotide sequence which includes the following elements, from N-terminal to C-terminal: (a) a scFV sequence which can bind specifically the idiotype of a human anti-gp120 antibody, (b) a hinge sequence and (c) a CH3 sequence. The minibodies can be expressed by a cell, a cell line or other suitable expression system. In some embodiments, a mammalian cell line (for example, CHO-K1 cell line) can be used as expression system to produce the minibodies, even bacterial expression systems (for example, E. coli, B. subtilis), yeast expression systems (for example, Pichia, S. cerevisiae) or any other expression system known to the person skilled in the art could be used.

According to an embodiment the minibody has the sequence SEQ ID NO:10 or SEQ ID NO:11 or a sequence identical at least by 95%, preferably at least by 98%, still more preferably by 99% to the sequence SEQ ID NO:10 o SEQ ID NO:11. Another object of the present invention is an immunogenic composition comprising an immunologically effective amount of at least an anti-idiotype minibody of the invention, preferably the minibody with VH and VL of P1 antibody described in patent EP2121763 according to the VH-VL configuration and a pharmaceutically acceptable carrier and/or diluent.

Optionally, the immunogenic composition can further include one or more adjuvants. An adjuvant is a compound which has a not specific stimulation activity on the immune system. Not limiting examples of adjuvants are immunostimulating complexes, saponins, mineral oils, vegetable oils, aluminium hydroxide, aluminium phosphate or aluminium oxide, etc.

Another object of the present invention is a kit of parts comprising an anti-idiotype minibody of the invention, and the gp120 HIV antigen or other natural or artificial antigens, as combined preparation for the simultaneous, separated or sequential administration in a regime of therapeutic or prophylactic immunization against HIV. At last, considering their capability to react specifically with anti-gp120/HIV antibodies, the anti-idiotype minibodies of the invention can be used as diagnostic reagents for detecting anti-gp120 antibodies and/or subpopulations of antibodies in a biological sample, such as for example a sample of serum, plasma, blood or any other suitable biological material derived from an animal, including a human being, for example a patient infected or suspected to be infected with HIV.

The present invention further relates to nucleotide sequences comprising or consisting of a sequence encoding an anti-idiotype minibody capable of specifically binding the idiotype of a human anti-gp120 antibody, preferably according to a VH-VL configuration, still more preferably said nucleotide sequence is an mRNA sequence. Advantageously, the mRNA sequence encoding for the minibody according to any one of the herein described embodiments can be used for generating messenger RNA (mRNA) vaccines with the advantage of being monocistronic. Systems for the in vivo administration of mRNA are described for example in Kauffman et al., 2016) (Guan & Rosenecker, 2017). In some embodiments said nucleotide sequences to ease the in vivo vehiculation thereof will be formulated in nanoparticles, for example in lipidic nanoparticles, cationic lipidic nanoparticles, example of such formulations can be found in US2020197510. According to an embodiment said mRNA encodes for a minibody according to any one of the herein described embodiments, for example for a minibody capable of specifically binding the idiotype of the human anti-gp120 b12 antibody. The present invention further relates to sequences of mRNA comprising or consisting of a sequence encoding an anti-idiotype minibody and immunogenic compositions including it. In an embodiment said nucleotide sequences are DNA sequences, for example SEQ ID NO:9 or coding nucleotide sequences SEQ ID NO:10.

The present invention further relates to vectors comprising molecules of nucleic acid encoding for minibodies according to any one of the herein described embodiments and the use thereof in the prevention and treatment of HIV infections. The gene encoding the minibody could be inserted in a vector which could further include sequences for controlling transcription and translation. Examples of Vectors which could be used are vectors of RNA viruses, vectors of DNA viruses, vectors of plasmid viruses, vectors of adenovirals, vectors of herpes virus and vectors of retrovirus. A further object of the present invention is a host cell transformed with one or more vectors capable of expressing the minibodies according to any one of the herein described embodiments.

### EXAMPLES AND EXPERIMENTAL DATA

### Drawing of P1 Minibody with Two Different Orientations

The minibody is an engineered form of immunoglobulins wherein the CH3 constant portion is added to ScFv format.

For drawing the minibody the article ‘Effective Therapeutic Approach for Head and Neck Cancer by an Engineered Minibody Targeting the EGFR Receptor’ Kim et al. PLoS One was taken into consideration (herein incorporated by reference). In details, in order to produce P1 minibodies with the two different orientations (VH-VL and VL-VH), the respective ScFvs were drawn. The variable sequences of light chain and heavy chain of the immunoglobulin denominated P1, anti-idiotype of the human b12 antibody, were taken into consideration and, between the two sequences, a sequence consisting of 18 aminoacidic residues (linker) corresponding to SEQ ID NO:3 was inserted. This linker sequence allows the three-dimensional assembly of the two chains, so that the binding capability of the original antibody is kept. The used linker was described in the work ‘An improved linker for single chain fv with reduced aggregation and enhanced proteolytic stability’ Whitlow et al. Protein Eng Des Sel 1993, wherein its capability of guaranteeing a greater solubility of the produced protein is demonstrated, which would favour the purification thereof.

In order to obtain the ScFvs of the minibodies, the hinge region (Hinge) was added, a flexible region present between the variable portion and the constant one of the antibodies, and the third portion of the constant region of the heavy chain (CH3, SEQ ID NO:8). Relatively to the CH3 portion, the sequence is murine (same original species of P1 antibody), and the sequence of the MAK33 antibody was used, since in literature ‘Folding and association of the antibody domain C(H)3: Prolyl isomerization preceeds dimerization’ M J Thies et al. J Mol. Biol. its crystallographic structure and homodimerization after expression and production in a prokaryotic system was described.

With the purpose of being able to clone these sequences in the expression vector (pCM) (‘A phage display vector optimized for the generation of human antibody combinatorial libraries and the molecular cloning of monoclonal antibody fragments’ Solforosi et al. New Microbiol), at 5′ the restriction site recognized by Sacl enzyme was inserted, whereas at 3′ the restriction site recognized by Spel enzyme was inserted. With the purpose of controlling that these restriction sites were not present inside the sequence, one proceeded with the restriction analysis of the sequences by the Plasma DNA tool. Since in P1 minibody with VH-VL orientation the restriction site was present even inside the sequence encoding the protein, the nucleotide sequence was modified through silent mutation. After having drawn these sequences, the corresponding DNA was made to be synthetized by a specialized firm.

### Cloning of P1 Minibodies with Different Orientation in the Expression Vector

The nucleotide fragment encoding for the P1 minibodies with different orientation and the expression vector (pCM) were digested with Sacl and Spel enzymes (New England Biolabs, NEB). The digestion reaction was performed for 1-2 h at 37° C., and the digestion product was analysed by electrophoretic run on 1% agarose gel.

Having verified the correct digestion, the bands of interest (or the bands of VH-VL P1 minibody gene and of VL-VH P1 minibody gene digested with Sacl and Spel with size of ˜1100 bp, and of the vector digested with Sacl and Spel with size of ˜4000 bp) were extracted from the agarose gel, by specific kit (QIAGEN), following the indications of the manufacturer.

Subsequently, one proceeded with ligations:
1) VH-VL P1 Mb gene digested with Sacl and Spel+vector digested with Sacl and Spel
2) VH-VL P1 Mb gene digested with Sacl and Spel+vector digested with Sacl and Spel

by using T4 ligase (NEB) and following the indications of the manufacturer. At the end, some microliters of the reaction were used to transform the XL1blue electrocompetent cells of E. coli strain (Stratagene). The next day 5 colonies were made to grow, for each transformation, at 37° C. under stirring overnight. From the growths the plasmid DNA was extracted, by using the specific kit (QIAGEN), which was sequenced to observe the correct cloning of DNA fragments.

### Expression and Purification of the P1 Minibodies with Different Orientation

The purification of the minibodies was performed by affinity chromatography.

In order to produce the P1 minibodies with different orientation, E. coli XL1blue electrocompetent cells were transformed with about 100 ng of (previously sequenced) vector. The next day a colony for each transformation was inoculated in 10 ml of SB with ampicillin and left to grow overnight under stirring at 37° C. The next morning, 5 ml of culture were subinoculated in 500 ml of SB with ampicillin at 37° C. under stirring and, when the cultures reached an optimum O.D.600 nm (≈0.6-0.8), they were induced with 250 ul of IPTG 1M and left under stirring at 30° C. overnight.

The next day, the cultures were centrifuged (15 minutes at 3100 rcf), the pellet was resuspended with 20 ml of PBS and subjected to 3 cycles of sonication (sonication 1′30″, rest 1′) in ice. The bacterial debris was removed by centrifugation (30 minutes at 10571 rcf). The supernatant is recovered and filtered by using a 0.2 μ-filter and one proceeds with the purification protocol by affinity chromatography by using the suitable resin (ThermoFisher) and following the indications of the manufacturer. The amount and purity degree of the purified proteins were evaluated by electrophoretic run on polyacrylamide gel.

### Evaluation in ELISA of B12 Bond Towards P1 Minibodies with Different Orientation

Once concluded the purification of P1 minibodies with different orientation, the affinity of the anti-HIV b12 antibody, in Fab format, towards P1 minibodies was evaluated by ELISA assay. In fact, since P1 is an internal image of b12 idiotype, the indispensable premise for an operation thereof as immunogen is the capability of recognizing the antibody itself.

On a 96-well plate (Corning) about 100 ng/well of the minibody on VH-VL orientation were bound and the plate was kept at 4° C. overnight.

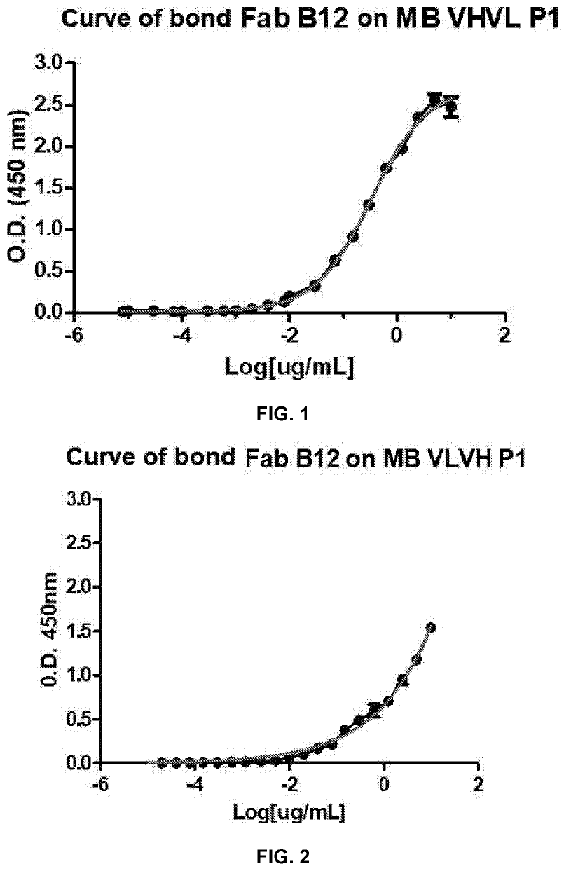

The next day, the plate was washed with H2O (so as to remove the exceeding antigen which did not bind), and it was blocked with a 1% PBS/BSA solution (Sigma) for 1 hour at 37° C. Once ended the incubation period, the 1% PBS/BSA solution was removed and dilutions of the anti-HIV Fab b12 antibody in base 2 (with 10 ng/μl) were added. The plate was incubated for 1 hour at 37° C. Once ended the incubation, the plate was washed 5 times with 0.1% PBS/Tween20 (Sigma) and the commercial secondary antibody was added, directed against the constant portion of the human light chains, conjugated with horseradish peroxidase (HRP, α-human Fab conjugated with HRP, Sigma). The plate was incubated for 45 minutes at 37° C. Once ended the incubation, the plate was washed 5 times with 0.1% PBS/Tween20 and 40 ul of the developing solution (TMB, 3,3′,5,5′-tetramethylbenzidine, Invitrogen, chromogenic substrate for detecting HRP) were added. After 15 minutes at 37° C., the reaction was blocked by adding 40 ul of stop solution (sulfuric acid, Carlo Erba). The colorimetric reading was performed by means of automatic reader of 450-nm plates (FIG. 1).

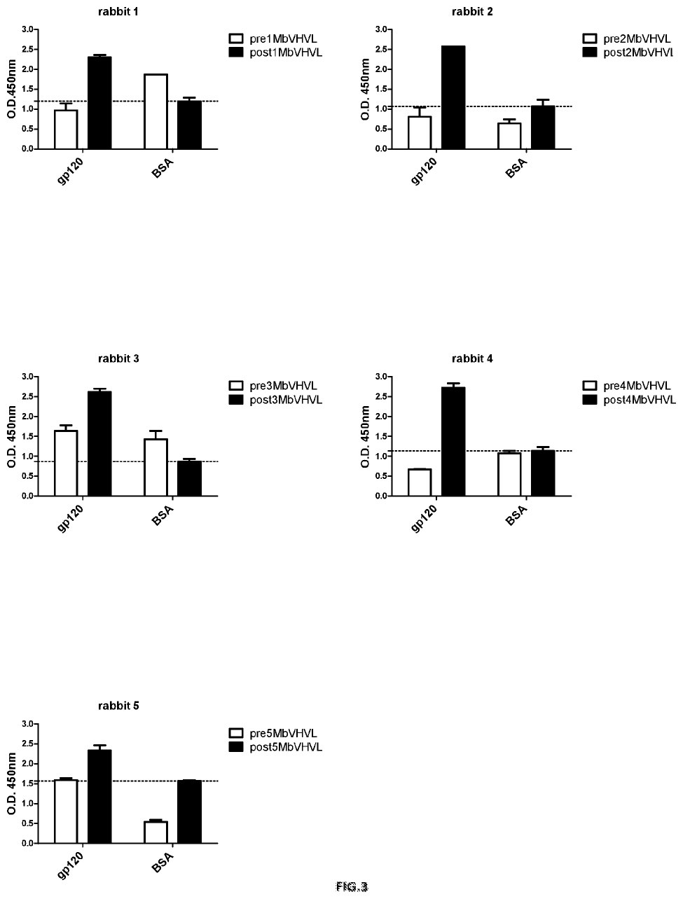

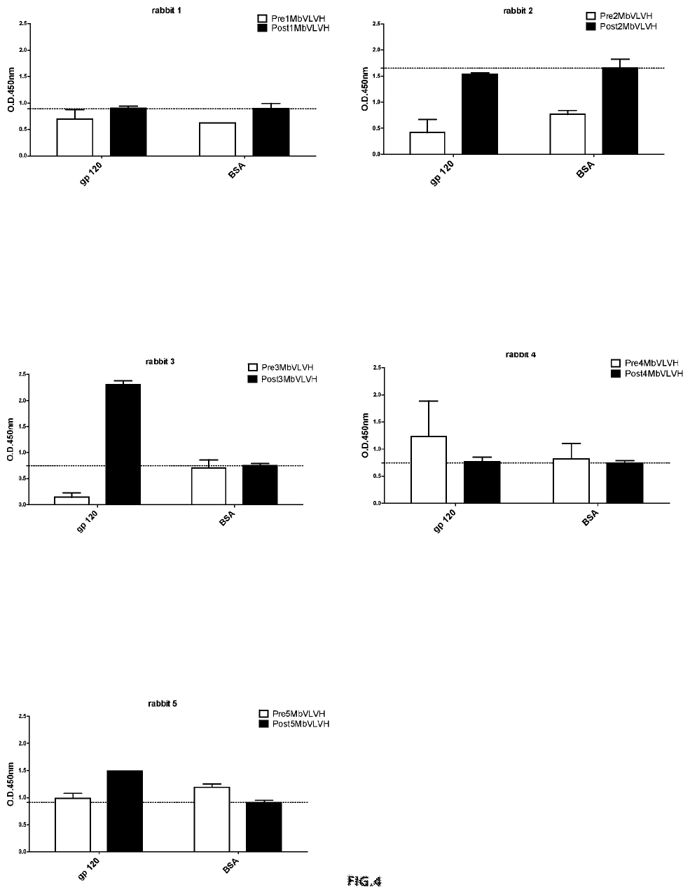

The same protocol was performed for evaluating the affinity of b12 antibody, in Fab format, towards the P1 minibody with VL-VH orientation. The only variation to the just described protocol relates the plate coating, wherein 100 ng/well of the P1 minibody with VL-VH orientation were coated. The results of this experiment are shown in FIG. 2.

### Use of the P1 Minibodies with Different Orientation as Immunogen in Rabbits

The P1 minibodies with different orientation were used as immunogens in rabbits in order to be able to evaluate their capability of inducing a specific immune response directed against gp120/HIV, the same antigen recognized by the b12 antibody which P1 is capable of causing after an immunization. For the immunization a protocol based upon the one described in ‘Anti-HIV-1 Response Elicited in Rabbits by Anti-Idiotype Monoclonal Antibodies Mimicking the CD4-Binding Site’ 2008 PLoS One′ was used.

In details, the immunization protocol performed by Davids Biotechnologie GmbH provided the use of cohorts, each one consisting of 5 rabbits (New Zealand White Rabbits). The protocol had 5 immunizations performed twice a week with 120 ug of immunogen (per rabbit) and as adjuvant the Addavax adjuvant was used. The overall duration of the protocol was 63 days, day of sacrifice of the different immunized rabbits.

The Table of the immunization protocol is shown herebelow.

Day
Order Confirmation: 3202

| | | |
|---|---|---|
| 22 Oct. 2018 | Preimmune | Day 1 |
| 22 Oct. 2018 | 1. Immunization | Day 1 |
| 5 Nov. 2018 | 2. Immunization | Day 14 |
| 19 Nov. 2018 | 3. Immunization | Day 28 |
| 26 Nov. 2018 | Test Bleed for ELISA Determination | Day 35 |
| 3 Dec. 2018 | 4. Immunization | Day 42 |
| 17 Dec. 2018 | 5. Immunization | Day 56 |
| 24 Dec. 2018 | Final Bleed | Day 63 |
| 7 Jan. 2019 | Estimated Shipping Date | |

The biological samples obtained by means of this experimentation were the following ones:
serum of rabbits before starting the immunization protocol (pre-immunization serum)
serum of rabbits collected during the immunization protocol, test-bleed, for evaluating the experimentation progress (it was observed if the serum collected on day 35 from the experimentation beginning was capable of reacting with the antigen used for immunization, to evaluate if the rabbit then actually was serum-positivizing for that antigen)
serum and spleen of the rabbits collected during and after sacrifice.

The immunization protocol was performed by using both P1 minibodies with different orientation, and a negative minibody (or a minibody which was not bound by the anti-HIV b12 antibody, in Fab format, produced and purified by following the same protocol used for P1 minibodies with different orientation).

### Evaluation of the Reactivity of Sera of Rabbits Towards gp120/HIV Glycoprotein

With the purpose of evaluating the capability of P1 minibodies with different orientation to induce in rabbits an immune response directed against gp120/HIV, an ELISA essay was performed by immobilizing on plate the gp120/HIV and by using as primary antibody different dilutions of serum of the immunized rabbits (as described in the article ‘Anti-HIV-1 Response Elicited in Rabbits by Anti-Idiotype Monoclonal Antibodies Mimicking the CD4-Binding Site’ 2008 PLoS One).

A 96-well plate (Corning) was coated with 100 ng/well of commercial gp120/HIV (strain YU2) and left at 4° C. overnight.

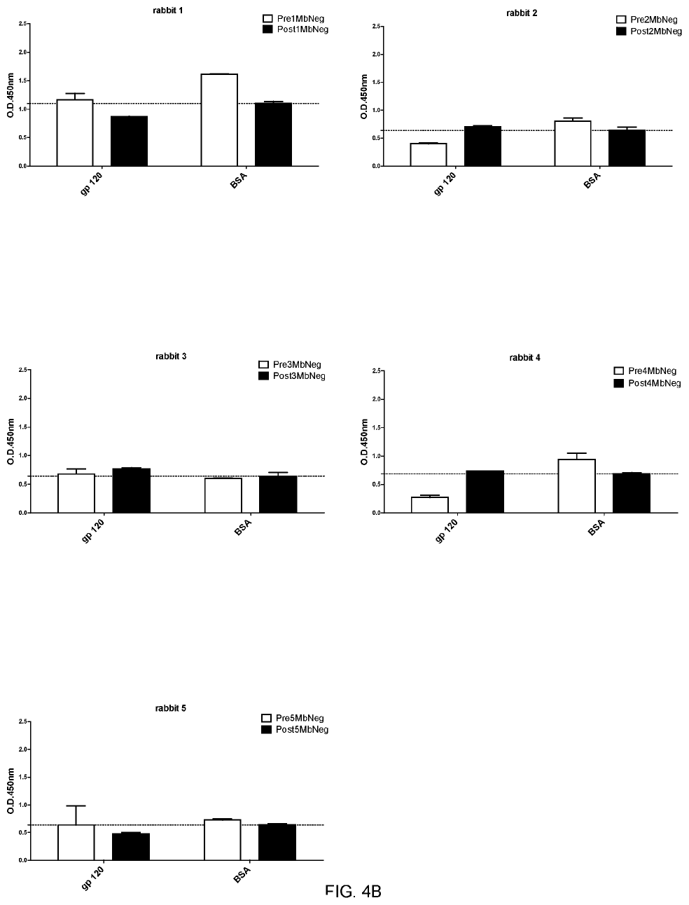

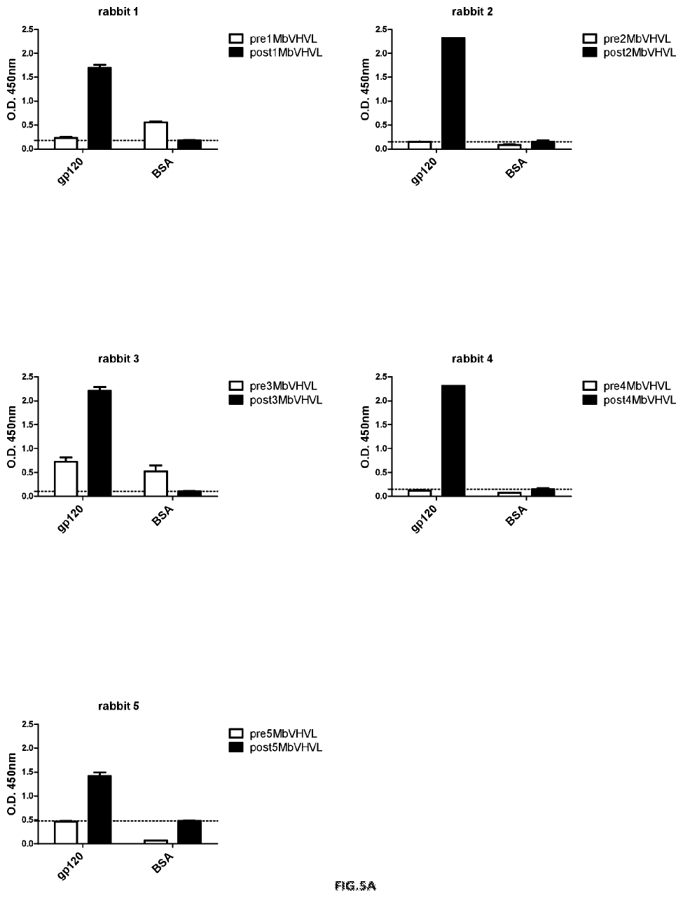

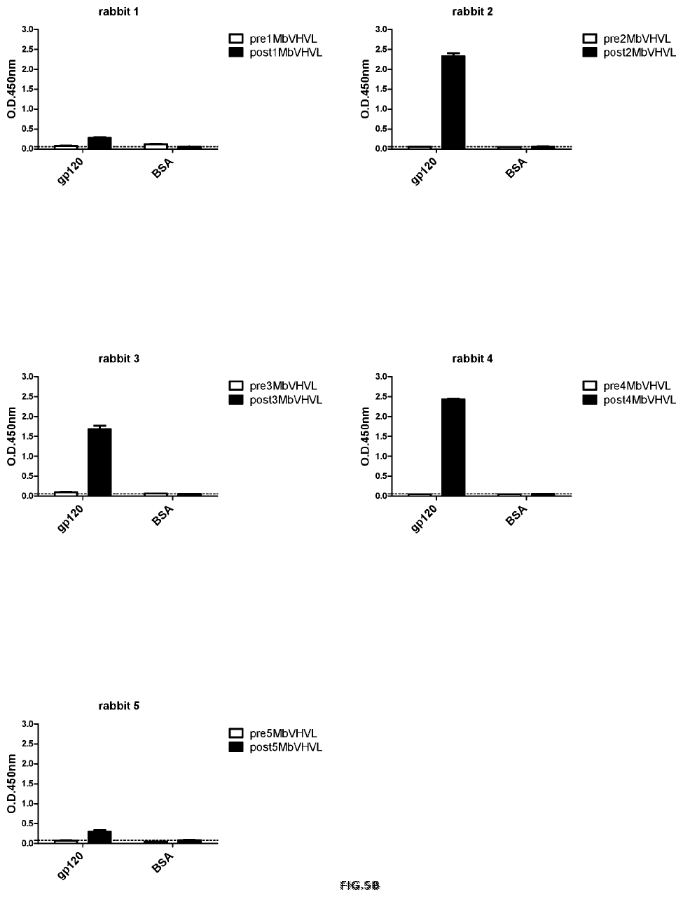

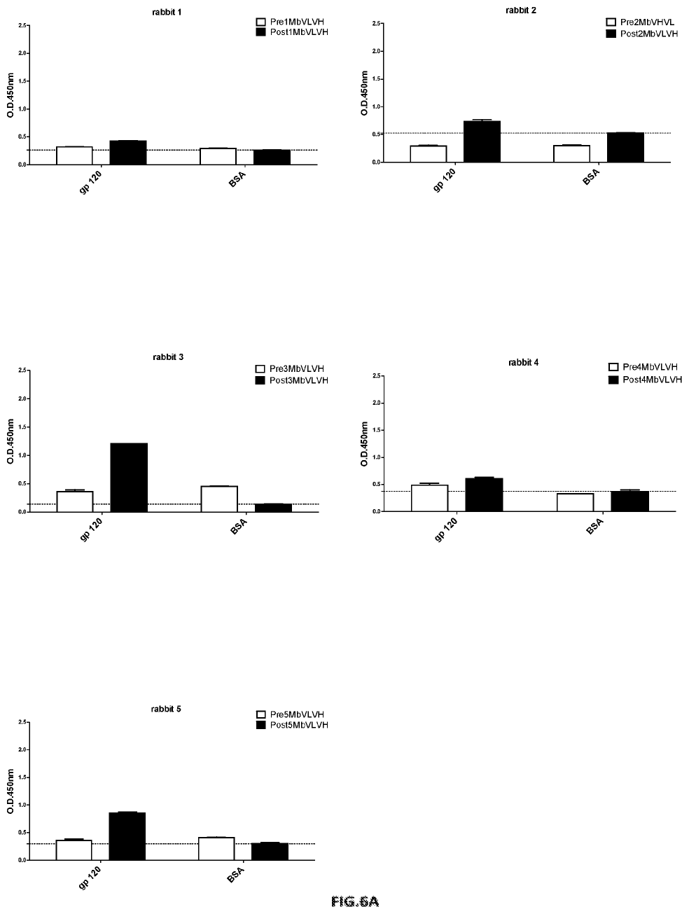

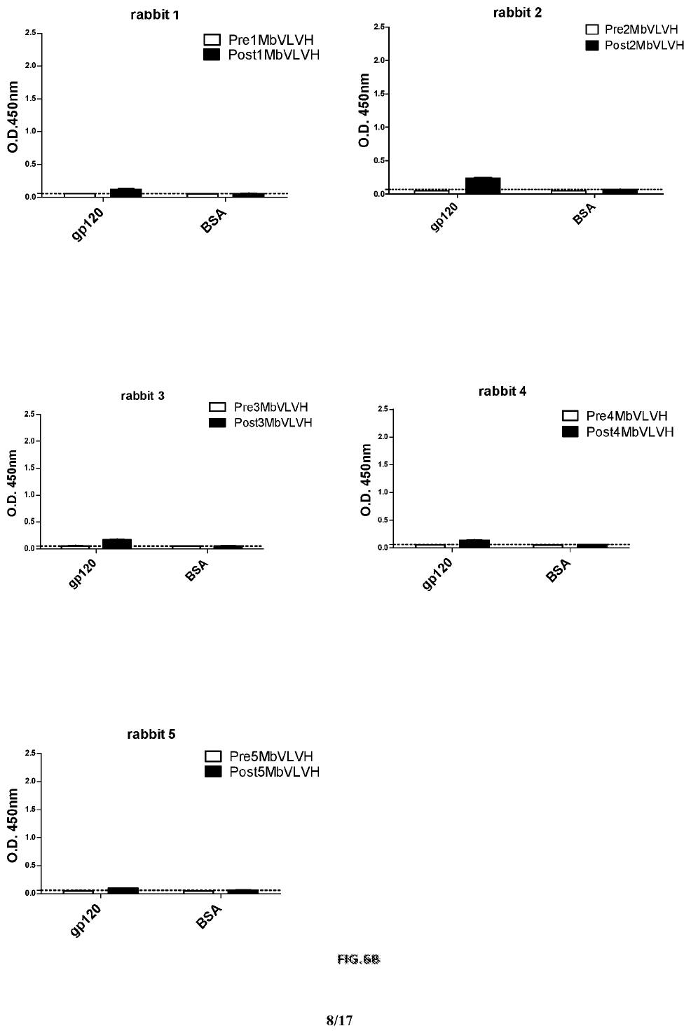

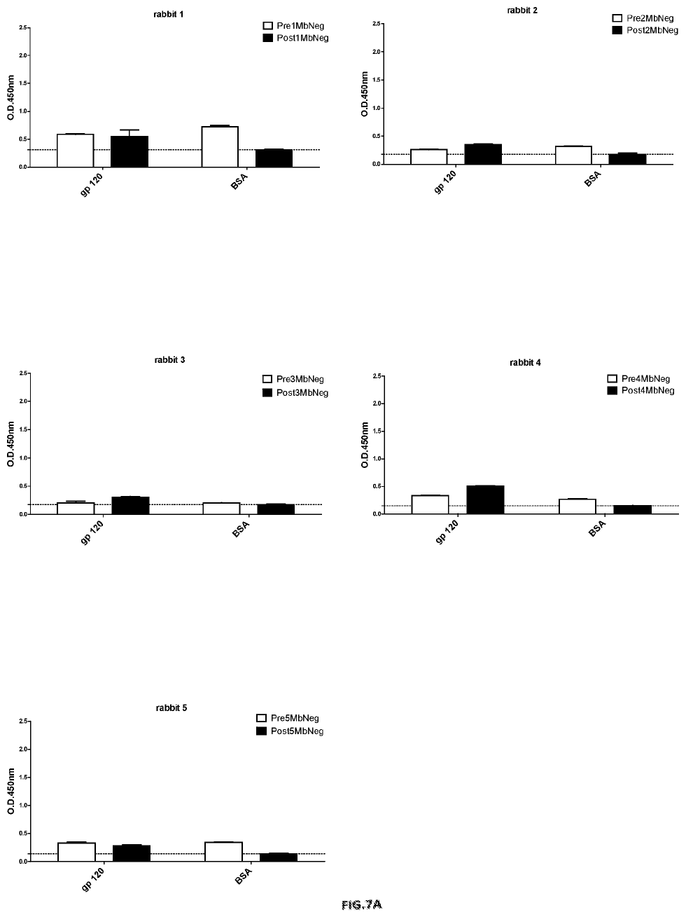

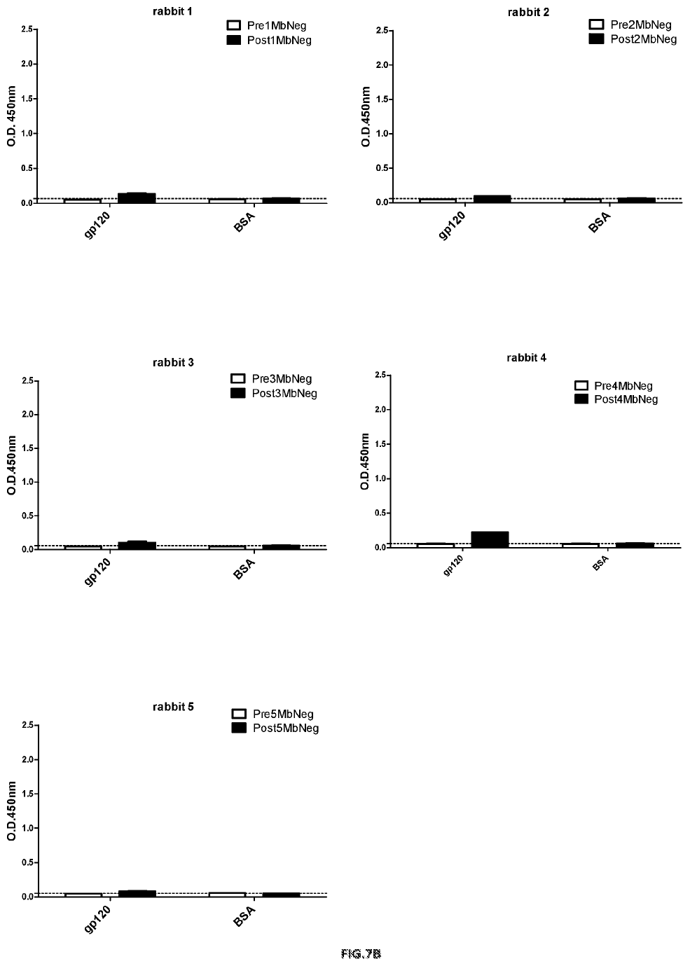

The next day, the plate was washed with H2O (so as to remove the exceeding antigen which did not bind) and blocked with a 1% PBS/BSA solution (Sigma) for 1 hour at 37° C. Once ended the incubation period, the 1% PBS/BSA solution was removed and the not diluted pre-immunization and post-immunization sera were added. The plate was incubated for 1 hour at 37° C. Once ended the incubation, 5 washing with 0.1% PBS/Tween20 (Sigma) were made and to each well 40 μl of the α-rabbit commercial secondary antibody were added, conjugated with horseradisch peroxidase (HRP, α-rabbit conjugated with HRP, Sigma). As positive control the anti-HIV b12 antibody (10 ng/μl) was used and, as control for possible nonspecific signals due to the secondary antibody, the α-rabbit secondary antibody was used on a well wherein the serum of rabbits was not added previously. The plate was left for 45 minutesat 37° C. Once ended the incubation, 5 washing with 0.1% PBS/Tween20 were made and to each well 40 ul of the developing solution (TMB, 3,3′,5,5′-tetramethylbenzidine, Invitrogen, chromogen substrate for detecting HRP) were added. After 15 minutes at 37° C., the reaction was blocked by adding 40 ul of stop solution (sulfuric acid, Carlo Erba). The colorimetric reading was performed by means of automatic reader of 450-nm plates. In order to evaluate possible nonspecific signals of sera, as control antigen BSA (Sigma) was used. The previously described protocol was used for evaluating sera obtained from rabbits immunized with P1 minibody with VH-VL orientation (FIG. 3), with P1 minibody with VL-VH orientation (FIG. 4) and with negative minibody (FIG. 4B). The same protocol was performed for evaluating the sera obtained from the rabbits diluted 1:20 and 1:200 immunized with P1 minibody VH-VL orientation (FIG. 5A/B), with P1 minibody with VL-VH orientation (FIG. 6A/B) and with negative minibody (FIG. 7A/B)

### Statistical Analysis

In order to evaluate if the signal obtained on gp120/HIV by using the serum of rabbits after immunization had a statistically significative relevance with respect to the signal obtained on the same protein by using the serum of the rabbits obtained before starting immunization, one proceeded with Wilcoxon analysis (not parametric test for not independent samples) by using the GraphPad Prism program.

This statistical analysis was made for the pre-and post-immunization serum of rabbits for each used immunogen and for the various dilutions taken into consideration in ELISA assay. Moreover, it has to be specified that the statistical significance considered for this test was p<0.1, due to the low power of the test, performed only on 5 rabbits which constituted the various immunization cohorts).

The Wilcoxon test was performed both by using the signals shown by the rabbit sera on gp120/HIV (as already described in literature ‘Epitopes for neutralizing antibodies induced by HIV-1 envelopeglycoprotein BG505 SOSIP trimers in rabbits and macaques’ P J Klasse et al. PLoS Pathog), and on the signal of gp120/HIV wherein the nonspecific signal obtained on BSA was previously subtracted from the same serum of the analysed rabbit.

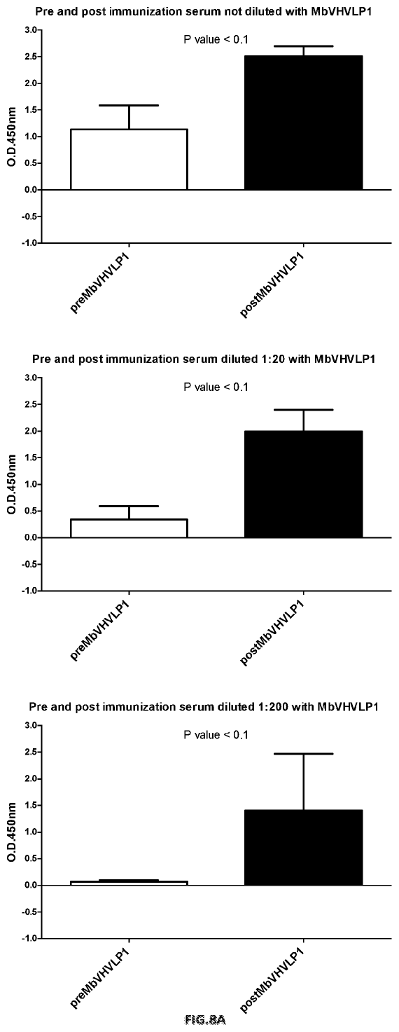

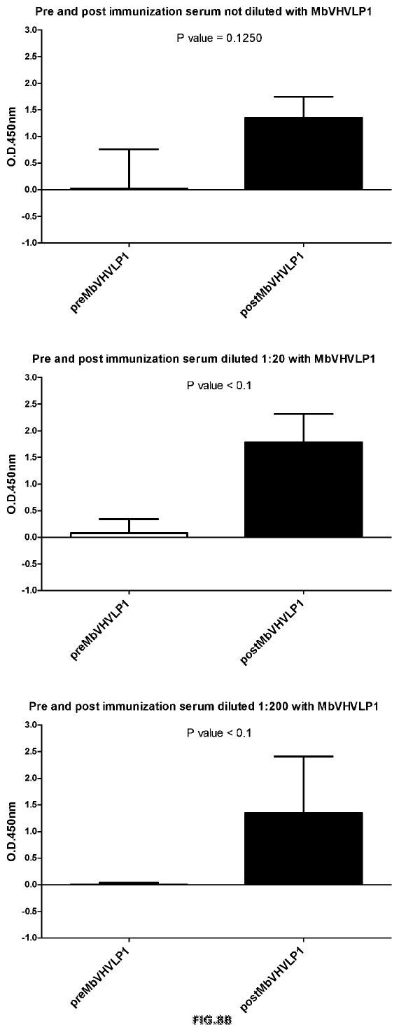

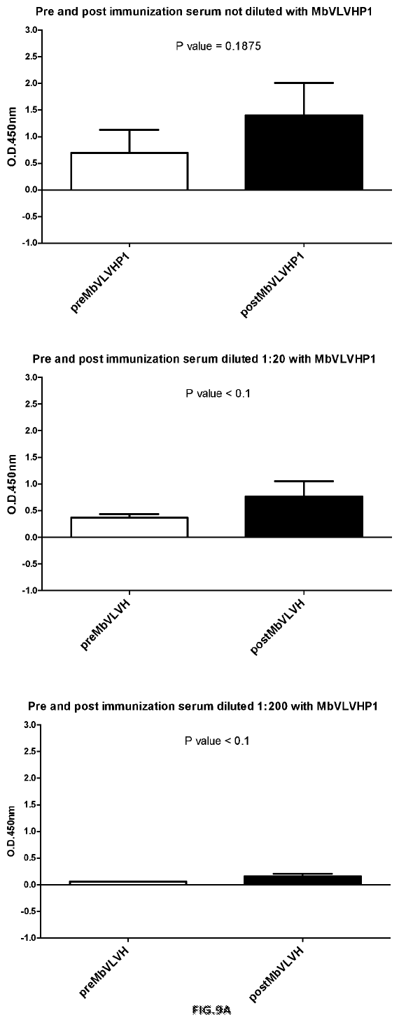

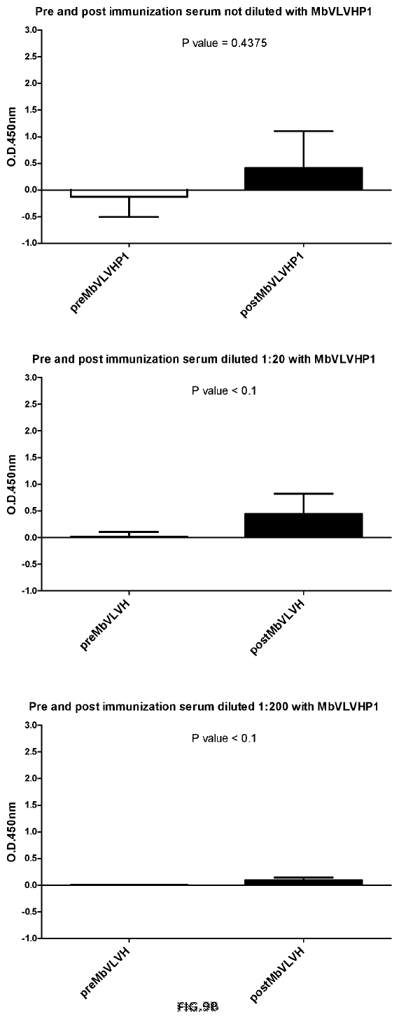

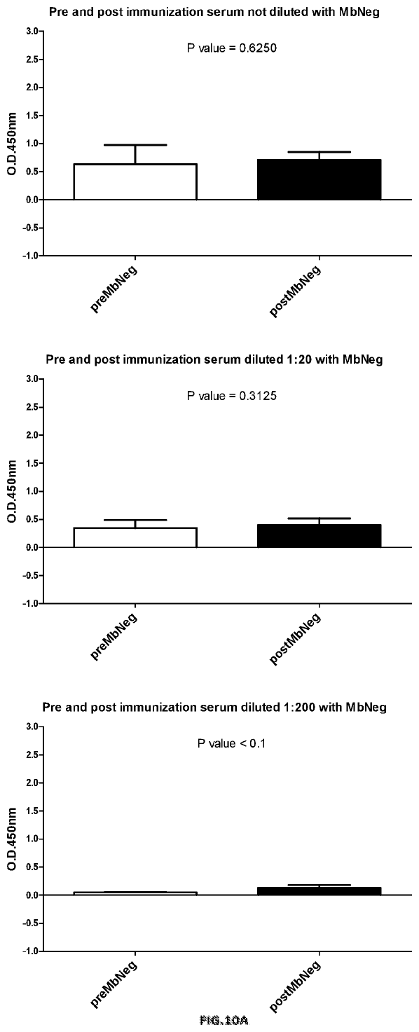

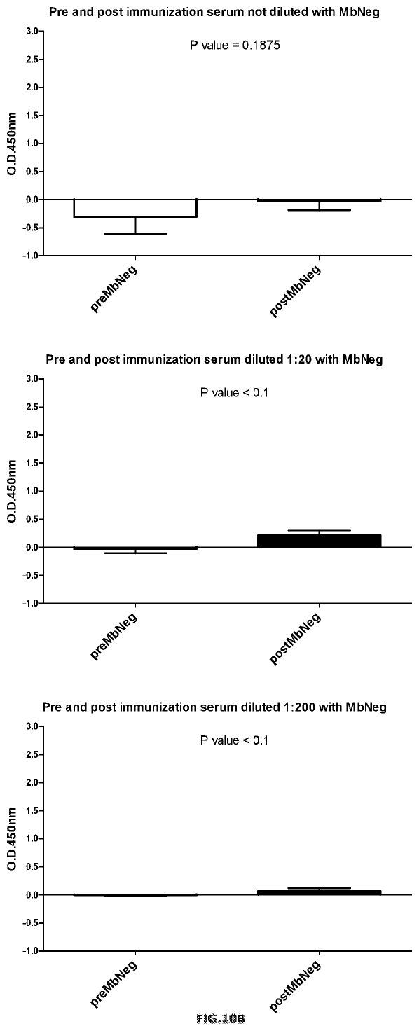

The graphs obtained by means of the statistical analysis performed for the various dilutions divided by the P1 minibody with VH-VL orientation (FIG. 8A/B), P1 minibody with VL-VH orientation (FIG. 9A/B) and negative minibody (FIG. 10A/B) are shown. In order to evaluate which one between the two minibodies was the best immunogen to induce a direct immune response against gp120/HIV, the statistical test of Mann-Withney (not parametric test for independent samples) was performed by using GraphPad Prism program.

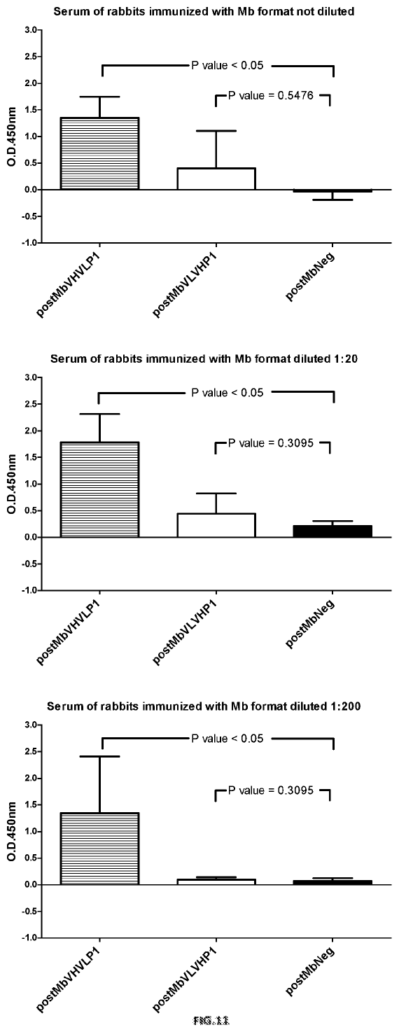

The statistical analysis was performed on the signal obtained on gp120/HIV, after having subtracted BSA, with the post-immunization sera of the rabbits divided by immunogen cohorts (FIG. 11).

### Conclusive Observations in the Light of the Obtained Experimental Data

The data in our possession show that:
by immunizing the rabbits with P1 minibody format with VH-VL orientation, molecule capable to react with the b12 antibody, an immune response directed against gp120/HIV developed.
by immunizing the rabbits with P1 minibody format with VL-VH orientation, molecule capable to react with the b12 antibody, a statistically significative immune response directed against gp120/HIV did not develop with respect to the one which develops by immunizing with the negative minibody.
by immunizing the rabbits with the negative minibody format, molecule not capable to react with the b12 antibody, an immune response directed against gp120/HIV did not develop.
by immunizing the rabbits with P1 minibody format with VH-VL orientation a statistically significative immune response directed against gp120/HIV develops with respect to the one which develops by immunizing with the negative minibody.

Based upon these observations, it can be concluded that the immunization with P1 minibody with VH-VL orientation demonstrated to be effective in stimulating an anti-anti idiotype response in rabbits, response which as expected is capable of recognizing the antigen recognized by the original idiotype (that of b12). On the contrary, P1 minibody format with VL-VH orientation, notwithstanding it reacts with the antibody 12 (then formally it is an anti-idiotype antibody), it is not capable of inducing the same type of response in rabbits. The two minibodies have identical, but inverted, sequence of the variable parts, they recognize the b12 epitope correctly, but only one of them succeeds in causing a statistically significative anti gp120 response.

### Description of Sequences

VL Amino acid sequence (P1 light variable chain)
SEQ ID NO: 1
E L V M T Q S P A S L A V S L G Q R A T I S C R A S Q S V S T S S Y S Y M H W
Y Q Q K P G Q P P K L L I K Y A S N L E S G V P A R F S G S G S G T D F T L N I
H P V E E E D T A T Y Y C Q H S W E I P Y T F G G G T K L E I K R
VH amino acid sequence (P1 heavy variable chain)
SEQ ID NO: 2
L E Q S G A E L V K P G A S V K I S C K A S G Y S F T D Y N M N W V K Q S N G
K S L E W I G V I N P N S G T T G Y N Q K F K G K A T L T V D Q S S S T A Y M Q
L N S L T S E D S A V Y Y C A E Y Y Y G E D P F A Y W G Q G T L V T V S T A
kttppsvyplapgsaaqt
Amino acid sequence linker 18 residues
SEQ ID NO: 3
G S T S G S G K P G S G E G S T K G
Amino acid sequence of ScFv P1 with VH-VL orientation
SEQ ID NO: 4
L E Q S G A E L V K P G A S V K I S C K A S G Y S F T D Y N M N W V K Q S N G
K S L E W I G V I N P N S G T T G Y N Q K F K G K A T L T V D Q S S S T A Y M Q
L N S L T S E D S A V Y Y C A E Y Y Y G E D P F A Y W G Q G T L V T V S T A G
S T S G S G K P G S G E G S T K G E L V M T Q S P A S L A V S L G Q R A T I S C
R A S Q S V S T S S Y S Y M H W Y Q Q K P G Q P P K L L I K Y A S N L E S G V
P A R F S G S G S G T D F T L N I H P V E E E D T A T Y Y C Q H S W E I P Y T F
G G G T K L E I K R
Amino acid sequence of I ScFv P1 with VL-VH orientation
SEQ ID NO: 5
E L V M T Q S P A S L A V S L G Q R A T I S C R A S Q S V S T S S Y S Y M H W
Y Q Q K P G Q P P K L L I K Y A S N L E S G V P A R F S G S G S G T D F T L N I
H P V E E E D T A T Y Y C Q H S W E I P Y T F G G G T K L E I K R G S T S G S G
K P G S G E G S T K G L E Q S G A E L V K P G A S V K I S C K A S G Y S F T D
Y N M N W V K Q S N G K S L E W I G V I N P N S G T T G Y N Q K F K G K A T L
T V D Q S S S T A Y M Q L N S L T S E D S A V Y Y C A E Y Y Y G E D P F A Y W
G Q G T L V T V S T A
Amino acid sequence of the hinge region
SEQ ID NO: 6
E P K S P K S A D K T H T A P
Amino acid sequence of CH3 of MAK33 (ID sequence 1CQK_A)
SEQ ID NO: 7
P A A P Q V Y T I P P P L E Q M A K D L V S L T C M I T D F F P E D I T V E W Q
W N G Q P A E N Y K N T Q P I M D T D G S Y F V Y S K L N V Q K S N W E A G
N T F T C S V L H E G L H N H H T E K S L S H
Amino acid sequence of the hinge region and amino acid sequence of
CH3 of MAK33
SEQ ID NO: 8
E P K S P K S A D K T H T A P P A A P Q V Y T I P P P L E Q M A K D L V S L T C
M I T D F F P E D I T V E W Q W N G Q P A E N Y K N T Q P I M D T D G S Y F V
Y S K L N V Q K S N W E A G N T F T C S V L H E G L H N H H T E K S L S H
Nucleotide sequence encoding the P1 minibody with VH-VL orientation
SEQ ID NO: 9
CTCGAGCAGTCTGGAGCTGAGCTGGTGAAGCCTGGCGCTTCAGTGAAGATATC
CTGCAAGGCTTCTGGTTACTCATTCACTGACTACAACATGAACTGGGTGAAGCA
GAGCAATGGAAAGAGCCTTGAGTGGATTGGAGTAATTAATCCTAACTCTGGTAC
TACTGGCTACAATCAGAAGTTCAAGGGCAAGGCCACATTGACTGTAGACCAAT
CTTCCAGCACAGCCTACATGCAGCTCAACAGCCTGACATCTGAGGACTCTGCA
GTCTATTACTGTGCAGAATATTACTACGGCGAGGATCCTTTTGCTTACTGGGGC
CAAGGGACTCTGGTCACTGTCTCTACAGCCGGCAGCACCAGCGGCAGCGGCA
AACCGGGCAGCGGCGAAGGCAGCACCAAAGGCGAACTCGTGATGACACAGTC
TCCTGCTTCCTTAGCTGTATCTCTGGGGCAGAGGGCCACCATCTCATGCAGGG
CCAGCCAAAGTGTCAGTACATCTAGCTATAGTTATATGCACTGGTACCAACAGA
AACCAGGACAGCCACCCAAACTCCTCATCAAGTATGCATCCAACCTAGAATCTG
GGGTCCCTGCCAGGTTCAGTGGCAGTGGGTCTGGGACAGACTTCACCCTCAA
CATCCATCCTGTGGAGGAGGAGGATACTGCAACATATTACTGTCAGCACAGTT
GGGAGATTCCGTACACGTTCGGAGGGGGGACCAAGCTGGAAATAAAACGGGA
ACCGAAAAGCCCGAAAAGCGCGGATAAAACCCATACCGCGCCGCCGGCGGCT
CCACAGGTGTACACCATTCCACCTCCCCTGGAGCAGATGGCCAAGGATCTAGT
CAGTCTGACCTGCATGATAACAGACTTCTTCCCTGAAGACATTACTGTGGAGTG
GCAGTGGAATGGGCAGCCAGCGGAGAACTACAAGAACACTCAGCCCATCATG
GACACAGATGGCTCTTACTTCGTCTACAGCAAGCTCAATGTGCAGAAGAGCAA
CTGGGAGGCAGGAAATACTTTCACCTGCTCTGTGTTACATGAGGGCCTGCACA
ACCACCATACTGAGAAGAGCCTCTCCCAC
Amino acid sequence of the P1 minibody with VH-VL orientation
SEQ ID NO: 10
L E Q S G A E L V K P G A S V K I S C K A S G Y S F T D Y N M N W V K Q S N G
K S L E W I G V I N P N S G T T G Y N Q K F K G K A T L T V D Q S S S T A Y M Q
L N S L T S E D S A V Y Y C A E Y Y Y G E D P F A Y W G Q G T L V T V S T A G
S T S G S G K P G S G E G S T K G E L V M T Q S P A S L A V S L G Q R A T I S C
R A S Q S V S T S S Y S Y M H W Y Q Q K P G Q P P K L L I K Y A S N L E S G V
P A R F S G S G S G T D F T L N I H P V E E E D T A T Y Y C Q H S W E I P Y T F
G G G T K L E I K R E P K S P K S A D K T H T A P P A A P Q V Y T I P P P L E Q
M A K D L V S L T C M I T D F F P E D I T V E W Q W N G Q P A E N Y K N T Q P I
M D T D G S Y F V Y S K L N V Q K S N W E A G N T F T C S V L H E G L H N H H
T E K S L S H
Nucleotide sequence encoding the P1 minibody with VL-VH orientation
SEQ ID NO: 11
GAGCTCGTGATGACACAGTCTCCTGCTTCCTTAGCTGTATCTCTGGGGCAGAG
GGCCACCATCTCATGCAGGGCCAGCCAAAGTGTCAGTACATCTAGCTATAGTT
ATATGCACTGGTACCAACAGAAACCAGGACAGCCACCCAAACTCCTCATCAAG
TATGCATCCAACCTAGAATCTGGGGTCCCTGCCAGGTTCAGTGGCAGTGGGTC
TGGGACAGACTTCACCCTCAACATCCATCCTGTGGAGGAGGAGGATACTGCAA
CATATTACTGTCAGCACAGTTGGGAGATTCCGTACACGTTCGGAGGGGGGACC
AAGCTGGAAATAAAACGGGGCAGCACCAGCGGCAGCGGCAAACCGGGCAGC
GGCGAAGGCAGCACCAAAGGCCTCGAGCAGTCTGGAGCTGAGCTGGTGAAGC
CTGGCGCTTCAGTGAAGATATCCTGCAAGGCTTCTGGTTACTCATTCACTGACT
ACAACATGAACTGGGTGAAGCAGAGCAATGGAAAGAGCCTTGAGTGGATTGGA
GTAATTAATCCTAACTCTGGTACTACTGGCTACAATCAGAAGTTCAAGGGCAAG
GCCACATTGACTGTAGACCAATCTTCCAGCACAGCCTACATGCAGCTCAACAG
CCTGACATCTGAGGACTCTGCAGTCTATTACTGTGCAGAATATTACTACGGCGA
GGATCCTTTTGCTTACTGGGGCCAAGGGACTCTGGTCACTGTCTCTACAGCCG
AACCGAAAAGCCCGAAAAGCGCGGATAAAACCCATACCGCGCCGCCGGCGGC
TCCACAGGTGTACACCATTCCACCTCCCCTGGAGCAGATGGCCAAGGATCTAG
TCAGTCTGACCTGCATGATAACAGACTTCTTCCCTGAAGACATTACTGTGGAGT
GGCAGTGGAATGGGCAGCCAGCGGAGAACTACAAGAACACTCAGCCCATCAT
GGACACAGATGGCTCTTACTTCGTCTACAGCAAGCTCAATGTGCAGAAGAGCA
ACTGGGAGGCAGGAAATACTTTCACCTGCTCTGTGTTACATGAGGGCCTGCAC
AACCACCATACTGAGAAGAGCCTCTCCCAC
Amino acid sequence of the P1 minibody with VL-VH orientation
SEQ ID NO: 12
E L V M T Q S P A S L A V S L G Q R A T I S C R A S Q S V S T S S Y S Y M H W
Y Q Q K P G Q P P K L L I K Y A S N L E S G V P A R F S G S G S G T D F T L N I
H P V E E E D T A T Y Y C Q H S W E I P Y T F G G G T K L E I K R G S T S G S G
K P G S G E G S T K G L E Q S G A E L V K P G A S V K I S C K A S G Y S F T D
Y N M N W V K Q S N G K S L E W I G V I N P N S G T T G Y N Q K F K G K A T L
T V D Q S S S T A Y M Q L N S L T S E D S A V Y Y C A E Y Y Y G E D P F A Y W
G Q G T L V T V S T A E P K S P K S A D K T H T A P P A A P Q V Y T I P P P L E
Q M A K D L V S L T C M I T D F F P E D I T V E W Q W N G Q P A E N Y K N T Q
P I M D T D G S Y F V Y S K L N V Q K S N W E A G N T F T C S V L H E G L H N
H H T E K S L S H
CDR1 VL
SEQ ID NO: 13
Q S V S T S S Y S Y
CDR2 VL (sequence 14 (tyr-ala-ser) was not inserted in the sequence
listing since it is a sequence lower than four residues)
SEQ ID NO: 14
Y A S
CDR3 VL
SEQ ID NO: 15
H S W E I P Y
CDR1 VH
SEQ ID NO: 16
G Y S F T D Y N
CDR2 VH
SEQ ID NO: 17
I N P N S G T T
CDR3 VH
SEQ ID NO: 18
A E Y Y Y G E D P F A Y

1. An anti-idiotype minibody comprising:
a scFV sequence capable of specifically binding the idiotype of a human anti-gp120 antibody, wherein said scFv comprises the variable domain of the heavy chain (VH) linked to the variable domain of the light chain (VL) with a linker sequence;
a hinge sequence; and
a IgG CH3 domain.

2. The minibody according to claim 1, wherein said scFv has a VH-VL configuration.

3. The minibody according to claim 1, wherein said scFv is capable of specifically binding the idiotype of the human anti-gp120 b12 antibody.

4. The minibody according to claim 1, wherein the idiotype of said human anti-gp120 antibody is directed against the portion of gp-120 that binds the CD4 receptor.

5. The minibody according to claim 1, wherein said minibody is capable of evoking an anti-gp120 immune response, in particular said minibody is murine.

6. The minibody according to claim 1, wherein the 3 CDRs of said variable domain of the heavy chain (VH) and the 3 CDRs of said variable domain of the light chain (VL) have the following amino acid sequences:
SEQ ID NO:13 Amino acid sequence CDR1 light chain
Tyr-Ala-Ser (YAS) Amino acid sequence CDR2 light chain
SEQ ID NO:15 Amino acid sequence CDR3 light chain
SEQ ID NO:16 Amino acid sequence CDR1 heavy chain
SEQ ID NO:17 Amino acid sequence CDR2 heavy chain and
SEQ ID NO:18 Amino acid sequence CDR3 heavy chain.

7. The minibody according to claim 1, wherein said variable domain of the heavy chain (VH) has the amino acid sequence SEQ ID NO:2 and said variable domain of the light chain (VL) has the amino acid sequence SEQ ID NO:1.

8. The minibody according to claim 1, wherein
said linker has the amino acid sequence SEQ ID NO:3; and/or
said hinge has the amino acid sequence SEQ ID NO:6; and/or
said igG domain has the amino acid sequence CH3 SEQ ID NO:7.

9. The minibody according to claim 1, wherein said scFV has the amino acid sequence SEQ ID NO:4 or SEQ ID NO:5 or a sequence at least by 95%, preferably at least by 98%, still more preferably by 99% to the sequence SEQ ID NO:4 or SEQ ID NO: 5.

10. The minibody according to claim 1 any one of claims 1 to 9, wherein said minibody has the amino acid sequence SEQ ID NO:10 or SEQ ID NO:12 or a sequence identical at least by 95%, preferably at least by 98%, still more preferably by 99% to the sequence SEQ ID NO:10 or SEQ ID NO:12.

11. A nucleotide sequence encoding a minibody according to claim 1, preferably said sequence is an mRNA or DNA sequence, in particular said nucleotide sequence having SEQ ID NO:9 or SEQ ID NO:11.

12. An expression vector comprising a nucleotide sequence according to claim 11.

13. The minibody according to, for use in the therapeutic or prophylactic treatment of HIV infection or related diseases, in particular by inducing a neutralizing immune response against HIV.

14. An immunogenic composition comprising the minibody according to claim 1 and a pharmaceutically acceptable carrier and/or diluent and/or adjuvant, in particular for use in the therapeutic or prophylactic treatment of HIV infection or related diseases.
